SEQUENCE LISTING (3 sequences)

SEQ ID NO: 1 - DNA, 256 bp, synthetic construct
  source 1..256: mol_type = other DNA
aagcttcagtgaattgtaatacgactcactatagggcgaattaagagagaaaagaagagtaagaagaaatataagacaccggtcgccaccatgggcctcacaggccactgaagtttatccatacttgacaccctggagggagctagcgtgaccagcggtagtctagaccttctgcggggcttgccttctggccatgcccttctctcccttgcacctgtacctcttggtctttgaataaagcctgagtaggctcgag

SEQ ID NO: 2 - DNA, 59 bp, synthetic construct
  source 1..59: mol_type = other DNA
gaagtgcagccggcgcgataggcctcacaggccagaagtcggcctcgaggcgaacattt

SEQ ID NO: 3 - DNA, 59 bp, synthetic construct
  source 1..59: mol_type = other DNA
gaagtgcagccggcgcgataggcctgtgaggccagaagtcggcctcgaggcgaacattt

(19) United States
(12) Patent Application Publication
(10) Pub. No.: US 2026/0317800 A1
(43) Pub. Date: Oct. 15, 2026

(54) METHODS AND COMPOSITIONS FOR SITE-SELECTIVE TERMINAL MODIFICATION OF NUCLEIC ACIDS, AND PRODUCTS THEREOF

(71) Applicant: The Hong Kong University of Science and Technology, Hong Kong (CN)

(72) Inventors: Yi KUANG, Hong Kong (CN); Chen CHENG, Hong Kong (CN); Zhenghua LIANG, Hong Kong (CN)

(21) Appl. No.: 19/641,605

(22) Filed: Apr. 8, 2026

Related U.S. Application Data
(60) Provisional application No. 63/785,583, filed on Apr. 8, 2025.

(51) Int. Cl. C12P 19/34

(52) U.S. Cl. CPC C12P 19/34

(57) ABSTRACT

Provided herein is a chemical method for site-selective terminal modification of a double-stranded nucleic acid molecule comprising (a) providing a double-stranded nucleic acid molecule comprising at least one single-stranded terminal overhang comprising a guanine base or guanine analog base; (b) contacting the double-stranded nucleic acid molecule with a carbonyl-introducing agent to provide an intermediate nucleic acid molecule comprising a carbonyl functional group at the guanine base or guanine analog base; and (c) contacting the intermediate nucleic acid molecule with an oxime-forming nucleophile to form a modified nucleic acid molecule comprising an oxime linkage at the guanine base or guanine analog base. Compositions containing reagents for performing the method, and products obtained thereby, are also provided. The disclosed method enables precise, enzyme-free terminal modification of linearized nucleic acids and allows installation of diverse chemical functionalities, facilitating downstream applications such as labeling, directional immobilization, and solid-phase in vitro transcription.

### CROSS-REFERENCE TO RELATED APPLICATIONS

The present application claims priority from U.S. Provisional Patent Application No. 63/785,583, filed on Apr. 8, 2025, which is hereby incorporated by reference in its entirety.

### REFERENCE TO SEQUENCE DISCLOSURE

The sequence listing file under the file name “107237US_SequenceListing.xml” submitted in ST.26 XML file format with a file size of 3,960 bytes created on Feb. 24, 2026 is incorporated herein by reference.

### TECHNICAL FIELD

The present disclosure pertains to the field of biotechnology and nucleic acid engineering. More specifically, the present disclosure relates to chemical methods and compositions for site-selective terminal modification of a single-stranded terminal overhang of double-stranded nucleic acids, products thereof, as well as applications of the same.

### BACKGROUND

Double-stranded DNA molecules, including plasmid DNA and other linear DNA constructs, are widely used in biotechnology, molecular biology, and nanotechnology. These nucleic acids often serve as templates for RNA synthesis, molecular probes, analytical tools, or structural elements in nanoscale assemblies. While DNA can frequently be used without modification, many applications require specific operations on one or both termini of a linear DNA molecule, such as the selective labeling or immobilization of the termini. For example, one end of a linearized plasmid can be anchored onto solid beads for repeated cycles of in vitro transcription.

Existing approaches for terminal DNA modification rely predominantly on enzyme-based methods, such as ligase-mediated attachment of chemically modified oligonucleotides or PCR amplification using chemically functionalized primers. Although PCR-based strategies allow sequence flexibility, they suffer from high cost, scalability constraints, and dependence on multiple enzymatic steps. Plasmid-based methods, on the other hand, offer higher yields and lower costs. However, they still require enzymes for terminal modifications, making them unsuitable for large-scale production due to high costs and dependence on complex workflows.

Accordingly, there remains a need for a cost-effective, enzyme-independent, and site-selective terminal modification method and relevant compositions capable of modifying the terminal overhang bases (such as a guanine base) of a double-stranded nucleic acid.

### SUMMARY

In a first aspect, the present disclosure provides a method for site-selective terminal modification of a double-stranded nucleic acid molecule, comprising: (a) providing a double-stranded nucleic acid molecule comprising at least one single-stranded terminal overhang containing a guanine base or guanine analog base; (b) contacting the double-stranded nucleic acid molecule with a carbonyl-introducing agent to provide an intermediate nucleic acid molecule comprising a carbonyl functional group at the guanine base or guanine analog base; and (c) contacting the intermediate nucleic acid molecule with an oxime-forming nucleophile to form a modified double-stranded nucleic acid molecule comprising an oxime linkage at the guanine base or guanine analog base.

In certain embodiments, the carbonyl-introducing agent has a structure of Formula I: R₁—CO—R₂, wherein R₁ and R₂ are independently selected from the group consisting of C₁-C₂₀ alkyl, C₂-C₂₀ alkenyl, C₃-C₁₂ cycloalkyl, C₁-C₂₀ epoxyalkyl, C₁-C₂₀ diazoalkyl, C₁-C₂₀ haloalkyl, aryl, heteroaryl, and aralkyl; and wherein at least one of R₁ and R₂ contains a leaving group selected from the group consisting of diazo, halogen, an epoxide group, and an activated Michael acceptor group.

In certain embodiments, the carbonyl-introducing agent is selected from the group consisting of α-diazocarbonyl compounds, phenacyl reagent, activated enone reagents, formylation reagents capable of installing a pendant aldehyde at the guanine base or guanine analog base, and aldehyde-transfer reagents capable of generating a pendant aldehyde at the guanine base or guanine analog base.

In certain embodiments, the one of R₁ and R₂ is selected from C₁-C₆ alkyl, and the other is selected from C₁-C₆ diazoalkyl.

In certain embodiments, the oxime-forming nucleophile has a structure of Formula II: R₃—O—NH—R₄, wherein R₃ and R₄ are independently selected from the group consisting of H, C₁-C₂₀ alkyl, C₁-C₂₀ haloalkyl, C₁-C₂₀ hydroxyalkyl, C₁-C₂₀ aminoalkyl, C₁-C₂₀ alkoxyalkyl, aryl, heteroaryl, a detectable label, an affinity tag, a polymer, a lipid, a solid support, an oligonucleotide, a reactive functional group, and pharmaceutically acceptable salts thereof.

In certain embodiments, the oxime forming nucleophile is a hydroxylamine-containing compound or an oxyamine-functionalized ligand, wherein the hydroxylamine-containing compound is selected from the group consisting of hydroxylamine, substituted hydroxylamines, O-substituted hydroxylamines, and pharmaceutically acceptable salts thereof; and wherein the oxyamine-functionalized ligand is selected from the group consisting of oxyamine-functionalized biotin derivatives, oxyamine-functionalized fluorophores, oxyamine-functionalized PEG reagents, oxyamine-functionalized polymers, oxyamine-functionalized lipids, oxyamine-functionalized oligonucleotides, oxyamine-functionalized solid supports, and pharmaceutically acceptable salts thereof.

In certain embodiments, the oxime-forming nucleophile is an O-substituted hydroxylamine, wherein R₃ is selected from the group consisting of C₁-C₂₀ alkyl, C₁-C₂₀ haloalkyl, C₁-C₂₀ hydroxyalkyl, C₁-C₂₀ aminoalkyl, PEG, aryl, heteroaryl, and an affinity tag, and wherein R₄ is H.

In certain embodiments, the oxime-forming nucleophile is an aminooxy functionalized ligand selected from the group consisting of aminooxy-functionalized biotin derivatives, aminooxy-functionalized fluorophores, aminooxy-functionalized PEG reagents, aminooxy-functionalized polymers, aminooxy-functionalized lipids, aminooxy-functionalized oligonucleotides, aminooxy-functionalized solid supports, and pharmaceutically acceptable salts thereof.

In certain embodiments, the oxime-forming nucleophile is hydroxylamine, Biotin-dPEG₁₁-oxyamine HCl, or BP FLUOR 488 Hydroxylamine, and wherein the carbonyl introducing agent is diazoacetone.

In certain embodiments, step (a) comprises providing a double-stranded nucleic acid molecule comprising two single-stranded terminal overhangs, wherein only one of the terminal overhangs contains a guanine base or a guanine analog base, and wherein the guanine analog base is a guanine derived or guanine related nucleobase capable of undergoing carbonyl installation and oxime formation, which is selected from the group consisting of 8-oxo-guanine, inosine, xanthine, 2-aminopurine, 7-deazaguanine, 8-bromoguanine, 6-thioguanine, O6-benzyl-guanine, 8-azido-guanine, iso-guanine, Z base, and any combination thereof.

In certain embodiments, the step (c) comprises attaching the oxime-forming nucleophile to a solid support selected from the group consisting of magnetic beads, polymeric beads, membranes, and microarray surfaces, prior to contacting the oxime-forming nucleophile with the intermediate nucleic acid molecule.

In certain embodiments, the oxime-forming nucleophile is part of a bifunctional linker comprising an aminooxy group and a second reactive group selected from the group consisting of azide, alkyne, maleimide, N-hydroxysuccinimide ester (NHS ester), and thiol; or, the double-stranded nucleic acid molecule is selected from the group consisting of DNA, cDNA, RNA, Locked Nucleic Acid (LNA), DNA/RNA hybrid, and derivatives thereof.

In certain embodiments, the step (a) comprises generating the single-stranded terminal overhang by an enzymatic method comprising digesting the double-stranded nucleic acid molecule with one or more restriction enzymes to generate at least one single-stranded terminal overhang containing an exposed guanine base or guanine analog base.

In certain embodiments, the one or more restriction enzymes are selected from the group consisting of Type II restriction enzymes, Type IIP restriction enzymes, Type IIS restriction enzymes, Type IIG restriction enzymes, Type III restriction enzymes, and any combination thereof.

In a second aspect, provided herein is a modified double-stranded nucleic acid molecule obtained by the method as described in the first aspect of the present disclosure.

In certain embodiments, the modified double-stranded nucleic acid molecule comprises a nucleic acid template configured for in vitro transcription (IVT).

In a third aspect, provided is a method for fluorescent labeling, protein-DNA conjugation, directional surface immobilization, biosensing, nanotechnology applications, or terminally controlled DNA cyclization or patterning, comprising applying the modified double-stranded nucleic acid molecule as described herein.

In a fourth aspect, provided herein is a kit for implementing the method for site-selective terminal modification of a double-stranded nucleic acid molecule, wherein the kit comprises: the carbonyl-introducing agent as described in the first aspect of the present disclosure; and the oxime-forming nucleophile as described in the first aspect of the present disclosure.

In certain embodiments, the kit further comprises one or more restriction enzymes, wherein the restriction enzymes are capable of digesting the nucleic acid to generate at least one single-stranded terminal overhang containing at least one exposed guanine base or guanine analog base; or wherein the kit further comprises a double-stranded nucleic acid molecule that is digestible by one or more restriction enzymes to generate one or two single-stranded terminal overhangs, wherein only one or both of the overhangs comprise a guanine base or guanine analog base.

In certain embodiments, the one or more restriction enzymes are selected from the group consisting of Type II restriction enzymes, Type IIP restriction enzymes, Type IIS restriction enzymes, Type IIG restriction enzymes, Type III restriction enzymes, and any combination thereof.

### BRIEF DESCRIPTION OF DRAWINGS

The above and other objects and features of the present disclosure will become apparent from the following description of the disclosure, when taken in conjunction with the accompanying drawings.

FIGS. 1A-1C illustrate an overview of the method as described herein. FIG. 1A: step 1, asymmetrical enzyme digestion to generate linearized plasmid DNA with single-stranded terminal overhangs, wherein one of the overhangs comprises a guanine base (G). FIG. 1B: step 2, site-specific carbonylation to introduce a C═O group at the O6 position of the exposed G base. FIG. 1C: step 3, oxime formation at the exposed G base.

FIGS. 2A-2B illustrates the design strategy for site-specific modification and verification thereof. FIG. 2A: the design strategy for site-specific fluorescent labeling of short double-stranded DNA using a BP Fluor 488 hydroxylamine dye. FIG. 2B: native polyacrylamide gel electrophoresis (PAGE) analysis before and after chemical treatments, demonstrating specific labeling at the sticky ends of short dsDNA, visualized through different channels. The image is overlay of GelRed channel (originally visualized in red) with Alexa Fluor 488 channel (originally visualized in green).

FIGS. 3A-3B illustrate a design strategy for site-specific modification with biotin on linearized plasmid and verification thereof. FIG. 3A: a design strategy for site-specific biotinylation of linearized plasmid DNA. Site-specific modification was verified by XhoI digestion and magnetic bead-based pull-down. FIG. 3B: native PAGE analysis showing the linearized plasmid and the distribution of XhoI-digested fragments, as well as the digested fragments remaining in the unbound fractions after biotinylation and/or streptavidin (DYNABEADS™ Streptavidin) pull-down.

FIGS. 4A-4B illustrate a design strategy for site-specific modification with biotin on linearized plasmid, followed by immobilization onto magnetic beads, and verification thereof. FIG. 4A: design strategy for site-specific terminal biotinylation followed by immobilization of the modified linearized plasmid on magnetic beads. FIG. 4B: validation of terminal modification with biotin occurring at either site-specific terminus. Agarose gel electrophoresis showing the presence of biotinylated or non-biotinylated plasmid DNA remaining in the unbound fractions after incubation with DYNABEADS™ Streptavidin beads.

FIG. 5 shows interaction with streptavidin to confirm the high yield of biotinylation on linearized plasmid. Agarose gel electrophoresis showing: (1) the plasmid DNA before incubation with DYNABEADS™ Streptavidin beads; (2) the plasmid DNA remaining in the unbound fractions after incubation with DYNABEADS™ Streptavidin beads (Unbound); and (3) the plasmid DNA recovered from the DYNABEADS™ Streptavidin beads (Bound).

FIG. 6 shows mRNA product analysis of in vitro transcription mRNA products generated by in vitro transcription using the biotinylated linearized plasmid templates prepared according to the method as described herein.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Throughout the present specification and claims, unless the context requires otherwise, the word “include” or variations such as “includes” or “including”, will be understood to imply the inclusion of a stated integer or group of integers but not the exclusion of any other integer or group of integers.

The methods or compositions of the present disclosure may comprise, consist essentially of, or consist of the steps or components/ingredients described herein.

The use of the singular herein includes the plural (and vice versa) unless specifically stated otherwise. For example, as used in this specification and the appended claims, the singular forms “a,” “an” and “the” can include plural referents unless the content clearly indicates otherwise.

In addition, where the use of the term “about” is before a quantitative value, the present teachings also include the specific quantitative value itself, unless specifically stated otherwise. As used herein, the term “about” refers to a ±10%, ±9%, ±8%, ±7%, ±6%, ±5%, ±4%, ±3%, ±2%, ±1%, or ±0% variation from the nominal value unless otherwise indicated or inferred.

Numeric ranges recited within the specification and appended claims are inclusive of the numbers defining the range and include each integer within the defined range. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1.5, and 4.25. This applies regardless of the breadth of the range.

As used herein, the term “and/or”, e.g., “A and/or B” shall be understood to mean either “A and B” or “A or B” and shall be taken to provide explicit support for both meanings or for either meaning, e.g. “A and/or B” includes the options i) “A”, ii) “B” or iii) “A and B”.

The term “optional(ly)” herein means that the feature (e.g., component, step, etc.) defined by the term may or may not be present. When the term is used in the claims and the specification, in the case where it indicates the “absence” of a defined feature, it should be interpreted as the claims and specification do not define the feature, rather than exclude the presence of the feature. For example, when the description or claims define the kit comprises “optionally, an instruction”, it involves two situations. The first situation indicates that the claims and specification define “the kit comprises an instruction”, and the other situation indicates that the claims and specification do not specifically define that “the kit comprises an instruction”, but do not exclude the existence of this feature, unless otherwise indicated in the context.

As used herein, the phrases “only one resulting overhang contains an exposed guanine base or guanine analog base,” “only one sticky end comprises a guanine base (e.g., an exposed guanine base),” and similar expressions refer to a double-stranded nucleic acid molecule in which, when one or two terminal overhangs are present, only one terminal overhang contains an exposed guanine base or guanine analog base while the opposite terminus, whether it presents a sticky end or a blunt end, does not contain an exposed guanine base or guanine analog base and therefore remains unreactive under the carbonyl-introducing and oxime-forming conditions described herein. It is worth noting that a nucleic acid comprising two single-stranded terminal overhangs, each of which may comprise one or more exposed guanine or guanine-analog nucleobases, is also contemplated herein.

As used herein, the term “nucleic acid” or “nucleic acid molecule” may be used interchangeably and refers to a natural or synthetic polymer of nucleotides including DNA, cDNA, synthetic dsDNA fragments, RNA, LNA, DNA/RNA hybrid, or derivatives thereof. In certain embodiments, the nucleic acid (molecule) is selected from the group consisting of linearized plasmid DNAs, PCR-amplified double-stranded DNA fragments, synthetic double-stranded DNA fragments, viral or bacteriophage-derived DNA fragments, and enzyme-processed DNA fragments. Unless the context indicates otherwise, the nucleic acid as described herein is double-stranded and can be linearized.

As used herein, “overhang”, “terminal overhang” or “single-stranded terminal overhang” may be used interchangeably and refers to a single-stranded extension at a terminus of a nucleic acid such as a double-stranded nucleic acid. Such an overhang may be generated enzymatically or chemically (e.g., by using Type-II or Type-IIS restriction enzymes, CRISPR-mediated cleavage followed by further processing, or adapter ligation). In certain embodiments, the overhang has a length of about 1-12 nt, for example, about 2-10 nt, about 2-8 nt, about 3-7 nt, about 3-5 nt, about 4-6 nt, or any individual numerical value or subranges therebetween.

As used herein, “exposed guanine base or guanine analog base” refers to a guanine base or guanine analog base located within a single-stranded terminal overhang of a double-stranded nucleic acid molecule, such that a carbonyl-introducing reaction can proceed at a suitable site thereof (e.g., the O6 position) under the applicable reaction conditions to introduce a carbonyl group on said guanine base or guanine analog base within the overhang. In certain embodiments, the overhang comprises one or more guanine bases or guanine analog bases. In certain embodiments, the guanine base or guanine analog base is disposed at the outermost end of the overhang.

As used herein, the term “carbonyl-introducing agent” refers to an electrophilic reagent capable of reacting with a guanine base or guanine analog base (e.g., at the O6 position) via e.g., O-alkylation or conjugate addition, thereby installing a pendant carbonyl-containing moiety at the guanine base or guanine analog base. The carbonyl-containing moiety may include, but is not limited to, a ketone group, an aldehyde group, or a carbonyl group present within a protected or masked carbonyl precursor. In certain embodiments, the carbonyl-introducing agent optionally comprises a leaving group, including diazo (—N₂), halogen (e.g., Cl, Br, I), epoxide, or an activated Michael acceptor group. The pendant carbonyl moiety installed at the guanine base or guanine analog base is capable of undergoing subsequent condensation with an oxime-forming nucleophile.

As used herein, the term “oxime-forming nucleophile” refers to a nucleophile comprising an aminooxy functionality (—O—NH₂ or —O—NHR, wherein R may be a hydrocarbyl or substituted hydrocarbyl, e.g., C₁-C₂₀ alkyl, C₁-C₂₀ haloalkyl, C₁-C₂₀ hydroxyalkyl, C₁-C₂₀ aminoalkyl, C₁-C₂₀ alkoxyalkyl, aryl, heteroaryl, or the like), hydroxylamine, or pharmaceutically acceptable salts thereof, that is capable of undergoing a condensation reaction with a ketone, aldehyde, or other carbonyl-containing moiety to form an oxime linkage. The nucleophile may be unsubstituted hydroxylamine, an O-substituted hydroxylamine of the formula R₃—O—NH₂ (wherein R₃ is as defined in formula II as described herein), or an aminooxy-functionalized molecule including, but not limited to, affinity tags, detectable labels, polymers, lipids, oligonucleotides, or solid supports bearing an aminooxy group.

As used herein, the term “site-selective terminal modification” refers to a chemical modification that occurs selectively at a guanine base or guanine analog base within a single-stranded terminal overhang of a double-stranded nucleic acid molecule, while the internal nucleotides remain base-paired and substantially unmodified under the reaction conditions. By the method as described herein, only the terminally exposed guanine base or guanine analog base undergoes carbonyl installation, thereby enabling site-selective (e.g., single-site) reactivity.

As used herein, the term “solid support” refers to any solid surface, particle, or matrix, including but not limited to magnetic beads, polymeric beads, membranes, microarrays, or chips. In certain embodiments, the solid support is optionally functionalized with streptavidin, NeutrAvidin, or another affinity ligand, thereby enabling immobilization of a terminal modified nucleic acid, such as an oxime-modified nucleic acid.

As used herein, the term “in vitro transcription (IVT) template” refers to a nucleic acid bearing a promoter (e.g., T7, T3, SP6) capable of directing RNA synthesis by an RNA polymerase.

As used herein, the “O6 position of guanine” refers to the oxygen atom of the carbonyl group (C═O) attached to the 6-carbon atom of the guanine purine ring, as shown in the structure of guanine set forth below:

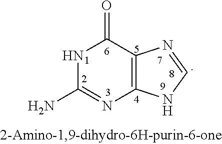 [SMILES: Nc1nc2[nH]cnc2c(=O)[nH]1]

This oxygen atom is a nucleophilic site and can serve as the target for O-alkylation or conjugate addition reactions with carbonyl-introducing agents as described herein. It should be noted that while the O6 position of an exposed guanine base is exemplified in some of the examples, other reactive positions of an exposed guanine base or a guanine-analog base are also amenable to the method as described herein.

As used herein, the term guanine-analog base refers to any purine-derived or purine-modified nucleobase that is structurally related to guanine and capable of forming a carbonyl intermediate suitable for oxime coupling. Such guanine analogs may include, but are not limited to, 8-oxo-guanine, inosine, xanthine, 2-aminopurine, 7-deazaguanine, 8-bromoguanine, 6-thioguanine, O6-benzyl-guanine, 8-azido-guanine, iso-guanine, Z-base, and other purine-based nucleobase analogs capable of undergoing carbonyl installation and oxime formation.

Unless otherwise specified, the term “C₁-C₆ hydrocarbyl” as used herein (including, but not limited to, C₁-C₆ alkyl, C₂-C₆ alkenyl, C₂-C₆ alkynyl, C₁-C₆ haloalkyl, C₁-C₆ hydroxyalkyl, and C₁-C₆ aminoalkyl) shall be understood to encompass, for each carbon number 1, 2, 3, 4, 5, or 6, the corresponding hydrocarbyl group and all structural isomers thereof. For example, when referring to the term “C₁-C₆ alkyl”, it shall be understood as separately describing all straight-chain, branched-chain, or cyclic alkyl groups having 1 to 6 carbon atoms (e.g., methyl, ethyl, n-propyl, isopropyl, n-butyl, isobutyl, sec-butyl, tert-butyl, pentyl, hexyl, and their isomers), and so on.

Unless defined otherwise, all technical and scientific terms used above have the same meaning as commonly understood by one of ordinary skill in the art to which embodiments of the present disclosure pertain.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

All publications, including all patents, patent applications and other patent and non-patent publications cited or mentioned herein are incorporated herein for reference only, and should not be construed as an admission of prior art.

In a first aspect, provided herein is a method for site-selective terminal modification of a double-stranded nucleic acid molecule, comprising providing a double-stranded nucleic acid molecule comprising at least one single-stranded terminal overhang(s) (e.g., one sticky end and one blunt end, or two sticky ends) containing a guanine base or guanine analog base (i.e., one or more exposed guanine bases or guanine analog bases); contacting the double-stranded nucleic acid molecule with a carbonyl-introducing agent to provide an intermediate nucleic acid molecule comprising a carbonyl functional group (e.g., a ketone or aldehyde functional group) at the guanine base or guanine analog base; and contacting the intermediate nucleic acid molecule with an oxime-forming nucleophile to form the modified nucleic acid molecule comprising an oxime linkage at the guanine base or guanine analog base.

In certain embodiments, the carbonyl-introducing agent has a structure of Formula I:

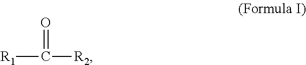

wherein R₁ and R₂ are independently selected from the group consisting of C₁-C₂₀ alkyl, C₂-C₂₀ alkenyl, C₃-C₁₂ cycloalkyl, C₁-C₂₀ epoxyalkyl, C₁-C₂₀ diazoalkyl, C₁-C₂₀ haloalkyl, aryl, heteroaryl, and aralkyl; and wherein at least one of R₁ and R₂ comprises a leaving group selected from diazo (—N₂), halogen (e.g., C₁, Br, I), an epoxide group (which is capable of nucleophilic ring-opening, thereby enabling installation of the carbonyl functional group at the guanine base or guanine analog base), and an activated Michael acceptor group.

In certain embodiments, the activated Michael acceptor group is selected from the group consisting of α,β-unsaturated ketones, α,β-unsaturated esters, α,β-unsaturated nitriles, α,β-unsaturated sulfones, and α,β-unsaturated sulfonamides. In certain embodiments, the activated Michael acceptor group is selected from methyl vinyl ketone, cyclohexenone, but-3-en-2-one, mesityl oxide, acrylonitrile, methyl acrylate, and vinyl sulfone.

In certain embodiments, the carbonyl-introducing agent is or comprises an α-diazocarbonyl compound of formula R₁—CO—CHN₂, wherein R₁ is defined as in Formula I. In certain embodiments, the carbonyl-introducing agent is or comprises an α-haloketone of formula R₁—CO—CH₂—X, wherein R₁ is defined as in Formula I and X is C₁, Br or I. In certain embodiments, R₁ is C₁-C₆ alkyl, C₂-C₆ alkenyl, C₃-C₆ cycloalkyl, aryl, heteroaryl, or aralkyl. In certain embodiments, R₁ is C₁-C₆ alkyl, C₂-C₆ alkenyl, or C₃-C₆ cycloalkyl. In certain embodiments, R₁ is methyl, ethyl, n-propyl, isopropyl, n-butyl, isobutyl, sec-butyl, tert-butyl, n-pentyl, isopentyl, neopentyl, n-hexyl, isohexyl, neohexyl, or straight-chain, branched or cyclic alkyl groups having 1 to 6 carbon atoms. In certain embodiments, R₁ is methyl, ethyl, or propyl.

In certain embodiments, the carbonyl-introducing agent is selected from the group consisting of α-diazocarbonyl compounds, such as diazoacetone, α-diazopropanone, α-diazoacetophenone, α-diazo-p-nitroacetophenone, or α-diazocyclohexanone); phenacyl reagents, such as phenacyl bromide, 4-nitrophenacyl bromide, 4-iodophenacyl bromide, or bis(trifluoromethyl) phenacyl bromide; activated enone reagents, such as methyl vinyl ketone, but-3-en-2-one, cyclohexenone, or mesityl oxide; formylation reagents capable of installing a pendant aldehyde at the guanine base or guanine analog base (e.g., at the O6 position); and aldehyde-transfer reagents capable of generating a pendant aldehyde at the guanine base or guanine analog base (e.g., at the O6 position).

In certain embodiments, the carbonyl-introducing agent is selected from diazo ketones, such as diazoacetone, diazoketone, α-diazocarbonyl compounds, α-diazopropanone, α-diazo-cyclohexanone, α-diazoacetophenone, or derivatives thereof.

In certain embodiments, one of R₁ and R₂ is selected from C₁-C₆ alkyl (e.g., methyl, ethyl, or propyl), C₂-C₆ alkenyl, and C₃-C₆ cycloalkyl, and the other is selected from C₁-C₆ diazoalkyl (e.g., diazomethyl, diazoethyl, diazopropyl, diazobutyl, diazopentyl, diazohexyl, or isomers thereof), C₁-C₆ epoxyalkyl, and C₁-C₆ haloalkyl. In certain embodiments, one of R₁ and R₂ is selected from C₁-C₆ alkyl, and the other is selected from C₁-C₆ diazoalkyl.

In certain embodiments, the oxime-forming nucleophile has a structure of Formula II:

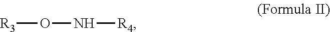

wherein R₃ and R₄ are independently selected from the group consisting of H, C₁-C₂₀ alkyl, C₁-C₂₀ haloalkyl, C₁-C₂₀ hydroxyalkyl, C₁-C₂₀ aminoalkyl, C₁-C₂₀ alkoxyalkyl, aryl, heteroaryl, a detectable label, an affinity tag, a polymer, a lipid, a solid support, an oligonucleotide, and a reactive functional group, wherein the oxime-forming nucleophile may be in the form of a pharmaceutically acceptable salt thereof.

In certain embodiments, R₃ and R₄ are independently selected from the group consisting of H, C₁-C₆ alkyl, C₁-C₆ haloalkyl, C₁-C₆ hydroxyalkyl, C₁-C₆ aminoalkyl, and C₁-C₆ alkoxyalkyl. In certain embodiments, one of R₃ and Ris H, and the other is selected from the group consisting of C₁-C₆ alkyl (e.g., methyl, ethyl, or propyl), C₁-C₆ haloalkyl, C₁-C₆ hydroxyalkyl, C₁-C₆ aminoalkyl, and C₁-C₆ alkoxyalkyl. In certain embodiments, both R₃ and R₄ are H.

In certain embodiments, the oxime forming nucleophile is selected from the group consisting of hydroxylamine-containing compound, such as hydroxylamine, substituted hydroxylamines, O-substituted hydroxylamines; oxyamine-functionalized ligand; aminooxy functionalized ligands; aminooxy polymers; aminooxy oligonucleotides; and pharmaceutically acceptable salts thereof.

In certain embodiments, the oxime-forming nucleophile is an O-substituted hydroxylamine, wherein R₃ is selected from the group consisting of C₁-C₂₀ alkyl (e.g., C₁-C₆ alkyl), C₁-C₂₀ haloalkyl (e.g., C₁-C₆ haloalkyl), C₁-C₂₀ hydroxyalkyl (e.g., C₁-C₆ hydroxyalkyl), C₁-C₂₀ aminoalkyl (e.g., C₁-C₆ aminoalkyl), PEG, aryl, heteroaryl, and an affinity tag, and wherein R₄ is H. In certain embodiments, the oxime-forming nucleophile is an oxyamine-functionalized ligand selected from the group consisting of oxyamine-functionalized biotin derivatives, oxyamine-functionalized fluorophores, oxyamine-functionalized PEG reagents, oxyamine-functionalized polymers, oxyamine-functionalized lipids, oxyamine-functionalized oligonucleotides, oxyamine-functionalized solid supports, and pharmaceutically acceptable salts thereof.

In certain embodiments, the oxime-forming nucleophile is an aminooxy functionalized ligand selected from the group consisting of aminooxy-functionalized biotin derivatives (e.g., biotin-ONH₂), aminooxy-functionalized fluorophores, aminooxy-functionalized PEG reagents, aminooxy-functionalized polymers, aminooxy-functionalized lipids, aminooxy-functionalized oligonucleotides, aminooxy-functionalized solid supports, and pharmaceutically acceptable salts thereof.

In certain embodiments, the oxime-forming nucleophile is hydroxylamine or a pharmaceutically acceptable salt thereof.

In certain embodiments, the oxime-forming nucleophile comprises an aminooxy-functionalized biotin derivative or an aminooxy-functionalized fluorescent dye. In certain embodiments, the biotin derivative comprises a polyethylene glycol (PEG) linker terminating in an aminooxy group. In certain embodiments, the fluorescent dye comprises a fluorophore bearing an aminooxy group configured for oxime formation. In certain embodiments, the oxime-forming nucleophile is Biotin-dPEG₁₁-oxyamine HCl or BP Fluor 488 Hydroxylamine.

In certain embodiments, the oxime-forming nucleophile is part of a bifunctional linker comprising an aminooxy group and a second reactive group selected from the group consisting of azide, alkyne, maleimide, NHS ester, and thiol.

In certain embodiments, the oxime-forming nucleophile is attached to a solid support selected from magnetic beads, polymeric beads, membranes, and microarray surfaces. In certain embodiments, step (c) comprises attaching the oxime-forming nucleophile to a solid support selected from magnetic beads, polymeric beads, membranes, and microarray surfaces prior to contacting it with the intermediate nucleic acid molecule.

It should be understood that any nucleic acid molecule capable of presenting a single stranded terminal overhang containing a guanine base or guanine analog base, while maintaining the remaining internal nucleotides in a double stranded and non-reactive conformation, is suitable for use in the method as described herein.

In certain embodiments, the double-stranded nucleic acid molecule is selected from the group consisting of DNA, cDNA, RNA, Locked Nucleic Acid (LNA), DNA/RNA hybrid, and derivatives thereof (either naturally occurred or synthesized). In certain embodiments, the double-stranded nucleic acid molecule is selected from the group consisting of linearized plasmid DNA, PCR-amplified double-stranded DNA fragments, synthetic double-stranded DNA fragments (e.g., gene fragments, GBLOCKS®, and other chemically synthesized dsDNA molecules of any length sufficient to maintain base-paired internal guanine bases), viral or bacteriophage-derived DNA fragments (provided they include a region suitable for introducing a unique guanine or guanine analog-containing overhang), and enzyme (e.g., SfiI, BglI, BsaI, BsmBI, SapI)-processed DNA fragments.

The nucleic acid molecule as described herein can be of any length, provided that all internal guanine bases remain base-paired under carbonylation conditions and shielded from electrophilic carbonylation while only the terminally exposed overhang guanine participates in the selective carbonylation reaction. In certain embodiments, the nucleic acid molecule has a length of at least 20 bp, at least 30 bp, at least 50 bp, at least 70 bp, at least 100 bp, at least 150 bp, at least 200 bp, at least 300 bp, at least 400 bp, at least 500 bp, e.g., at least 1 kb, at least 1.5 kb, at least 2 kb, at least 2.5 kb, or at least 3 kb (so long as the internal guanine bases of the nucleic acid molecule are base-paired and unreactive under the reaction conditions of step (b)).

In certain embodiments, step (a) comprises generating the single-stranded terminal overhang by an enzymatic, chemical, or synthetic method. In certain embodiments, step (a) comprises generating the single-stranded terminal overhang by an enzymatic method comprising digesting the nucleic acid molecule with one or more restriction enzymes (i.e., one or more restriction endonucleases) so as to generate at least one single-stranded terminal overhang containing at least one (such as one, two, or more) exposed guanine base or guanine analog base. In certain embodiments, the one or more restriction enzymes are selected from the group consisting of symmetrical restriction enzymes and asymmetrical restriction enzymes. In certain embodiments, the one or more restriction enzymes are selected from the group consisting of Type II restriction enzymes (e.g., SfiI, BglI, BamHI, NotI, KpnI, and PstI), Type IIP restriction enzymes, Type IIS restriction enzymes (e.g., BsaI, BsmBI, SapI), Type IIG restriction enzymes, Type III restriction enzymes. In certain embodiments, the one or more restriction enzymes may be identical or non-identical and/or may recognize asymmetrical or symmetrical recognition sequences, and any combination thereof. In certain embodiments, the one or more restriction enzymes are selected from the group consisting of BsaI, BsmBI, SapI, SfiI, BglI, BamHI, NotI, KpnI, PstI, BglII, HindIII, NcoI, and any combination thereof.

In certain embodiments, the nucleic acid molecule comprises two sticky ends, wherein only one of the sticky ends comprises a (e.g., one, two, three, or more) guanine base or guanine analog base. In certain embodiments, the nucleic acid molecule comprises one sticky end, and one blunt end, wherein the sticky end comprises a (e.g., one, two, three, or more) guanine base or guanine analog base. In certain embodiments, the nucleic acid molecule comprises two sticky ends, wherein both sticky ends comprise a (e.g., one, two, three, or more) guanine base or guanine analog base.

In certain embodiments, the carbonyl-introducing reaction in step (b) takes place at any suitable nucleophilic position (e.g., the N2, O6, or N7 position) of the guanine base or guanine analog base.

In certain embodiments, the nucleic acid molecule is a linearized plasmid NDA comprising two sticky ends (i.e., single-stranded overhangs), wherein only one of the sticky ends or both ends comprise a guanine base or guanine analog base. In certain embodiments, one of R₁ and R₂ of the carbonyl-introducing agent is selected from C₁-C₆ alkyl, and the other is selected from C₁-C₆ diazoalkyl (e.g., diazoacetone); and one of R₃ and R₄ of the oxime-forming nucleophile is H, and the other is selected from the group consisting of H and C₁-C₆ alkyl (e.g., methyl, ethyl, or propyl). In certain embodiments, the carbonyl-introducing agent is diazoacetone, and the oxime-forming nucleophile is a hydroxylamine containing compound such as hydroxylamine, Biotin-dPEG₁₁-oxyamine HCl (BIOTIN-DPEG®₁₁-oxyamine HCl), or BP Fluor 488 Hydroxylamine.

In certain embodiments, the guanine analog base is a guanine derived or guanine related nucleobase capable of undergoing carbonyl installation and oxime formation, which is selected from the group consisting of 8-oxo-guanine, inosine, xanthine, 2-aminopurine, 7-deazaguanine, 8-bromoguanine, 6-thioguanine, O6-benzyl-guanine, 8-azido guanine, iso-guanine, Z base, and any combination thereof.

In a second aspect, provided herein is a modified double-stranded nucleic acid molecule obtained by the method of the method described herein (e.g., the method of any embodiments of the first aspect of the present disclosure).

In certain embodiments, the modified double-stranded nucleic acid molecule comprises a nucleic acid template configured for in vitro transcription. In certain embodiments, the modified double-stranded nucleic acid molecule comprises a T7, T3, or SP6 promoter, thereby serving as an in vitro transcription (IVT) template.

In certain embodiments, the modified double-stranded nucleic acid molecule comprises an additional element to facilitate subsequent applications. The element may include, but is not limited to, a label (e.g., a fluorophore or fluorescent label), a biotin moiety, a lipid, a linker, or a solid support such as beads or magnetic beads.

In a third aspect, provided is a method for fluorescent labeling, protein-DNA conjugation, directional surface immobilization, biosensing (e.g., surface plasmon resonance (SPR) and atomic force microscopy (AFM)), nanotechnology applications, or terminally controlled DNA cyclization or patterning, comprising applying the modified double-stranded nucleic acid molecule as described herein.

In a fourth aspect, provided herein is a kit or composition for implementing the method for site-selective terminal modification of a double-stranded nucleic acid molecule (such as the method as described in the first aspect herein), wherein the kit or composition comprises: the carbonyl-introducing agent as defined in the first aspect herein; the oxime-forming nucleophile as defined in the first aspect herein.

In certain embodiments, the kit or composition further comprises one or more restriction enzymes and/or a first double-stranded nucleic acid molecule (e.g., a dsDNA molecule), wherein the restriction enzymes are capable of digesting the nucleic acid to generate at least one (e.g., one or two) single-stranded terminal overhangs, wherein only one of the resulting sticky ends or both ends comprise an exposed guanine base or guanine analog base. In certain embodiments, the first double-stranded nucleic acid molecule is an unmodified nucleic acid molecule (e.g., linearized double-stranded DNA, plasmid DNA, etc.). In certain embodiments, the enzymes are selected from the group consisting of Type II restriction enzymes (e.g., SfiI, BglI, BamHI, NotI, KpnI, and PstI), Type IIP restriction enzymes, Type IIS restriction enzymes (e.g., BsaI, BsmBI, SapI), Type IIG restriction enzymes, Type III restriction enzymes, and any combination thereof. In certain embodiments, the one or more restriction enzymes may be identical or non-identical and/or may recognize symmetrical or asymmetrical recognition sequences, and any combination thereof. In certain embodiments, the restriction enzymes are selected from the group consisting of BsaI, BsmBI, SapI, SfiI, BglI, BamHI, NotI, KpnI, PstI, BglII, HindIII, NcoI, and any combination thereof.

In certain embodiments, the kit or composition further comprises a second double-stranded nucleic acid molecule comprising one or two single-stranded terminal overhang(s) (e.g., one sticky end and one blunt end, or two sticky ends), wherein only one overhang or both overhangs comprise a guanine base or guanine analog base (e.g., one or more guanine bases, preferably one guanine base).

In certain embodiments, the first double-stranded nucleic acid molecule (e.g., a dsDNA molecule) comprises a restriction site or sequence segment that is specifically recognized and digested by one or more restriction enzymes so as to generate one or two single-stranded terminal overhangs, wherein only one resulting overhang or both overhangs contain an exposed guanine base or guanine analog base. In certain embodiments, the first and/or second double-stranded nucleic acid molecule further comprises a template region and/or one or more promoters (e.g., T7, T3, or SP6 promoters) configured for in vitro transcription.

In certain embodiments, the kit further comprises an instruction for selectively modifying a nucleic acid molecule so that it comprises a guanine or guanine analog base-containing terminal overhang according to the method as described in the first aspect herein.

In certain embodiments, one of R₁ and R₂ of the carbonyl-introducing agent is selected from C₁-C₆ alkyl, and the other is selected from C₁-C₆ diazoalkyl (e.g., diazoacetone); and one of R₃ and R₄ of the oxime-forming nucleophile is H, and the other is selected from the group consisting of H and C₁-C₆ alkyl (e.g., methyl, ethyl, or propyl). In certain embodiments, the carbonyl-introducing agent is diazoacetone, and the oxime-forming nucleophile is a hydroxylamine group containing compound such as hydroxylamine, Biotin-dPEG₁₁-oxyamine HCl (such as BIOTIN-DPEG®₁₁-oxyamine HCl), or BP FLUOR 488 Hydroxylamine.

In a fifth aspect, provided herein is use of the modified double-stranded nucleic acid molecule obtained by the method described herein (e.g., the method of any embodiments of the first aspect of the present disclosure).

In certain embodiments, the modified double-stranded nucleic acid molecule is used as or used in a member selected from the group consisting of: an in vitro transcription template;

a fluorescently labeled nucleic acid; a protein-DNA conjugate; a directionally surface-immobilized DNA construct for biosensing; a DNA construct for use in surface plasmon resonance (SPR), atomic force microscopy (AFM), or nanotechnology applications; and an end-modified DNA construct suitable for terminally controlled cyclization or patterning. In certain embodiments, the modified double-stranded nucleic acid molecule is used as a nucleic acid template for in vitro transcription, e.g., for in vitro RNA transcription.

In a sixth aspect, provided herein is a solid-phase IVT template comprising the nucleic acid of any aspects of the present disclosure (e.g., a nucleic acid obtained by the method of the first aspect of the present disclosure) immobilized on a magnetic or polymeric bead.

In certain embodiments, the solid-phase IVT template is reusable for at least two, three, five, or ten or more (such as 10-100, 20-70, 30-60, 40-50, 60-80) rounds of in vitro transcription.

In certain specific embodiments, the method for site-selective terminal modification of a double-stranded nucleic acid molecule (e.g., linearized plasmids) as described herein comprises the following steps:

Step (a1): generation of linearized nucleic acid comprising one or two sticky ends (i.e., single-stranded overhangs), wherein only one sticky end or both ends comprise one or more exposed guanine bases or guanine analog base. For example, this step can be carried out by introducing at least one asymmetrical restriction enzyme site into the nucleic acid, such that digestion with one or more restriction enzymes yields two distinct single-stranded terminal overhangs. The restriction enzymes may be selected such that only one of the terminal overhangs contains an exposed guanine base or guanine analog base, while the opposite overhang lacks guanine bases or guanine analog base and therefore remains unreactive under the subsequent chemical modification conditions. In certain embodiments, the restriction enzymes are selected such that both of the terminal overhangs contain an exposed guanine base or guanine analog base.

Step (b1): Carbonyl-installation (e.g., acetonylation reaction). Subjecting the linearized nucleic acid to a carbonyl-introducing reaction, such as an acetonylation reaction, at the terminal overhang that contains the exposed guanine base or guanine analog base. In certain embodiments, the carbonyl-introducing agent selectively installs a carbonyl functional group at the guanine base or guanine analog base (e.g., at the O6 position thereof), thereby forming a carbonyl-modified intermediate suitable for subsequent oxime ligation.

Step (c1) Oxime formation. Reacting the carbonyl-modified nucleic acid with an oxime-forming nucleophile, such as hydroxylamine or an O-substituted hydroxylamine (e.g., aminooxy-functionalized reagents), to form a stable oxime linkage at the terminal guanine base or guanine analog base. This step may comprise installing a desired functional group, label, linker, or affinity handle at a single, site-selective position.

By using the method, composition, or kit as described herein, the present invention addresses the limitations of current enzyme-dependent methods and provides a number of significant advantages over traditional enzyme-based terminal modification approaches, for example:

Enzyme-free terminal modification. The chemical strategy eliminates the dependency on enzymes (such as ligases, polymerases, and other costly enzymes) traditionally required for nucleic acid terminal modification, thereby reducing cost, avoiding enzyme-related inefficiencies, and simplifying the overall workflow.

Exceptional scalability for industrial production. Because the method is fully chemical and does not depend on enzymatic conversion efficiency, it is inherently scalable and well-suited for industrial-scale operations, such as large-batch preparation of modified plasmids and DNA templates, RNA vaccine production.

Cost-effectiveness and reduced reliance on specialized reagents. The method avoids the need for chemically modified primers, engineered oligonucleotides, or specialized enzyme formulations, drastically lowering production and material costs.

True site-selectivity enabled by terminal-overhang design. Only the terminally exposed guanine or guanine analog base in a single-stranded overhang undergoes carbonylation and oxime formation, while all internal guanine bases or guanine analog bases remain double-stranded and unreactive. This ensures site-selective terminal modification, enabling directional immobilization, eliminating off-target reactions and minimizing non-specific labeling.

Versatility in downstream applications. The method allows the attachment of a variety of chemical groups (including fluorophores, biotin, PEG linkers, lipids, polymers, and solid supports), offering broad applicability for downstream applications, including fluorescent labeling, protein-DNA conjugation, biosensing, nanotechnology, and controlled DNA cyclization and patterning, RNA synthesis, molecular diagnostics, and functional assays.

Highly efficient solid-phase in vitro transcription (IVT) and template reuse. Terminal biotinylation via oxime chemistry allows DNA templates to be immobilized on magnetic or polymeric beads, and reused for multiple rounds of IVT, significantly improving yield and lowering operational cost.

Together, these advantages make the present method a robust, scalable, and cost-effective chemical platform for site-selective terminal modification of double-stranded nucleic acids, especially linearized plasmids used in RNA production and biotechnology applications.

The scope of the present disclosure is defined by the appended claims, along with the full scope of equivalents to which such claims are entitled. It should be appreciated that various modifications, equivalents, and substitutions may be made to the aspects and/or embodiments disclosed herein without departing from the spirit and scope of the invention, and such modifications, equivalents, and substitutions are intended to be within the scope of the present disclosure.

Following are examples that illustrate procedures for practicing the invention. These examples should not be construed as limiting.

### EXAMPLES

### Example 1. Site-Specific Terminal Modification of a Plasmid DNA

Table 1 shows the sequences inserted into the plasmid vector, which were selected to introduce specific restriction enzyme sites into an existing plasmid containing a transcription template.

TABLE 1
Insert sequences and corresponding restriction enzyme sites. Underlined
bases represent restriction enzyme recognition sites, and bases shown in
bold italics indicate specially designed base distributions.

| Name | Insert Sequence (5′-3′) | Enzyme site |
|---|---|---|
| Insert_1 (SEQ ID | AAGCTTCAGTGAATTGTAATACGACTCACTATAGG | GGCCTCACA |
| NO: 1) | GCGAATTAAGAGAGAAAAGAAGAGTAAGAAGAA | GGCC |
| | ATATAAGACACCGGTCGCCACCATGGGCCTCACAG | |
| | GCCACTGAAGTTTATCCATACTTGACACCCTGGAG | |
| | GGAGCTAGCGTGACCAGCGGTAGTCTAGACCTTCT | |
| | GCGGGGCTTGCCTTCTGGCCATGCCCTTCTCTCCC | |
| | TTGCACCTGTACCTCTTGGTCTTTGAATAAAGCCTG | |
| | AGTAGGCTCGAG | |
| SfiI/BglI Insert_2 | GAAGTGCAGCCGGCGCGATAGGCCTCACAGGCCA | GGCCTCACA |
| (SEQ ID NO: 2) | GAAGTCGGCCTCGAGGCGAACATTT | GGCC |
| SfiI/BglI Insert_3 | GAAGTGCAGCCGGCGCGATAGGCCTGTGAGGCCA | GGCCTGTGA |
| (SEQ ID NO: 3) | GAAGTCGGCCTCGAGGCGAACATTT | GGCC |

The recombinant plasmid was synthesized by Azenta (China), expressed in Escherichia coli cells at 37° C. and 220 rpm overnight, and extracted and purified using the TIANprep Plasmid Midi Kit (TIANGEN, China).

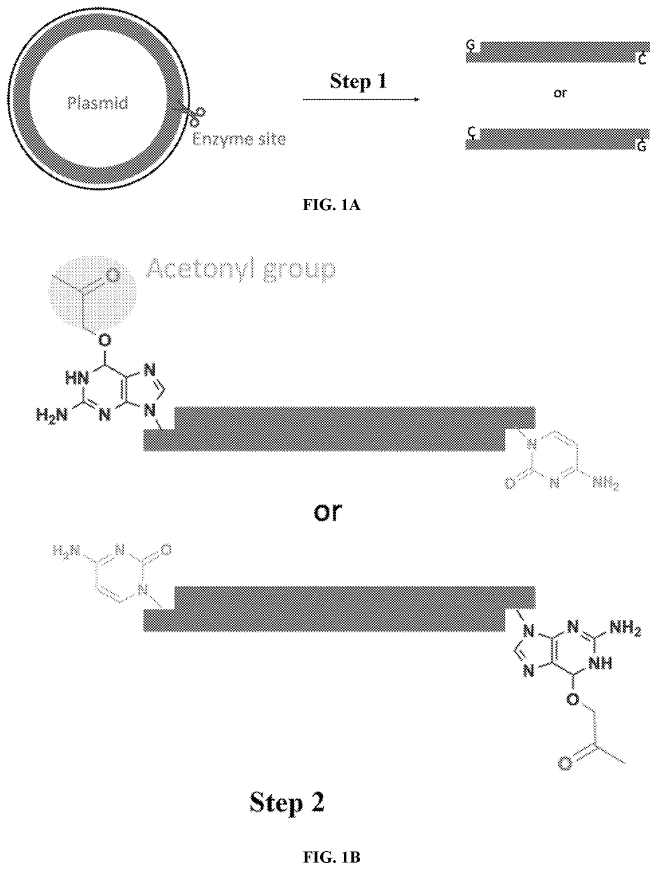

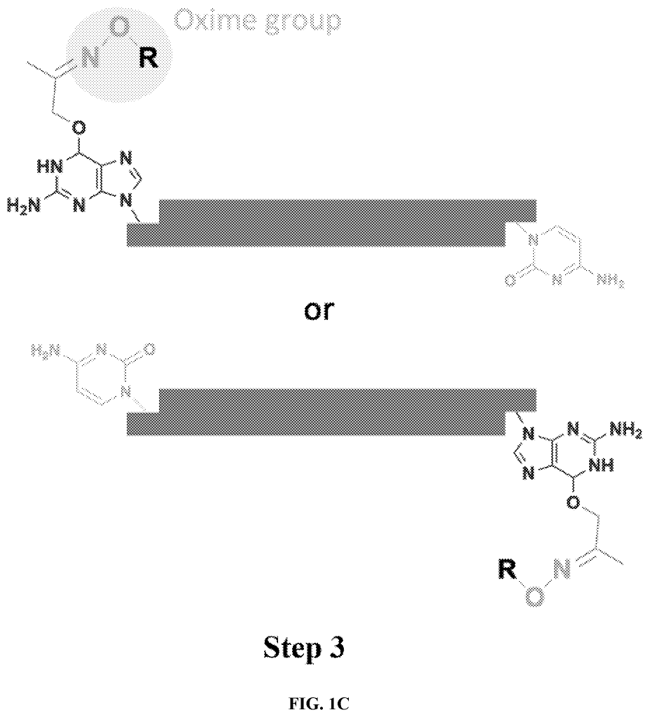

The inserted sequence contains recognition sites for either SfiI (GGCCTCAC↓AGGCC, incubated at 50° C. for 2 hours) or BglI (GCCTCAC↓AGGC, incubated at 37° C. for 2 hours). These enzymes linearize the plasmid DNA, exposing G bases at the 5′ sticky ends while leaving the 3′ sticky ends without G bases. By modifying the portion of the sequence shown in bold italics in Table 1, the distribution of exposed G bases can be adjusted (FIG. 1A). Linearized plasmid templates were purified using the QIAquick PCR Purification Kit (Qiagen, Germany).

After linearization, the two-step chemical reaction selectively modifies the exposed G bases at the sticky end of the linearized plasmid template (FIGS. 1B-1C). Table 2 lists the chemical reagents used in the reaction, including diazoacetone (provided by Prof. ZHOU Yuanyuan at The Hong Kong University of Science and Technology) and Biotin-dPEG-oxyamine (Merck, Germany).

TABLE 2
Structural formulas of the reactants used in the reactions

| Name | Structure |
|---|---|
| Diazoacetone | 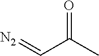 |
| BP Fluor 488 Hydroxylamine dye | 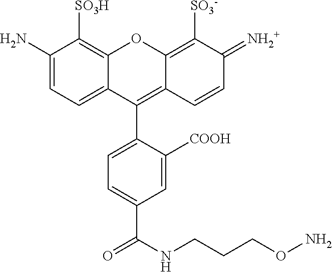 |
| BIOTIN-DPEG®₁₁- oxyamine HCl | 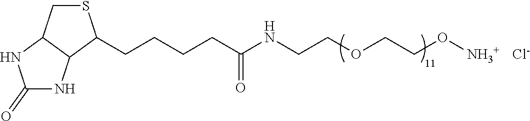 |

Table 3 shows the reaction cocktail for acetonylation, including the linearized and purified DNA template, magnesium acetate (Mg(OAc)₂), 2-(N-morpholino) ethanesulfonic acid (MES) buffer, and dichloro(1,5-cyclooctadiene)dirhodium(I) dimer ([Rh(COD)Cl]₂). The reaction mixture (10 μL) was incubated at room temperature for 30 minutes to allow the addition of the acetonyl group to the exposed G bases. The reaction product was purified using the QIAquick PCR Purification Kit (Qiagen, Germany). The detailed synthesis mechanism is shown in FIG. 1B.

TABLE 13
Acetonylation reaction cocktail

| | Stock | | Final |
|---|---|---|---|
| Materials | concentration | Volume | concentration |
| DNA template | 487 ng/μL | 4.1 μL | 200 ng/μL |
| Mg(OAc)2 | 50 mM | 2 μL | 10 mM |
| MES buffer | 100 mM, pH 6.0 | 2 μL | 20 mM |
| Diazoacetone in 10% | 400 mM | 1 μL | 40 mM |
| tetrahydrofuran (THF) | | | |
| [Rh(COD)Cl]2 in THF | 5.56 mM | 0.9 μL | 0.5 mM |
| Total volume | | 10 μL | |

Table 4 shows the reaction cocktail for oxime formation. After acetonylation, the reaction mixture (50 μL) was incubated at room temperature for 2 hours to allow the addition of the biotin group to the DNA template. The product templates were purified using the same method as described above. The detailed synthesis mechanism is shown in FIG. 1C.

TABLE 4
Oxime formation reaction cocktail

| | Stock | | Final |
|---|---|---|---|
| Materials | concentration | Volume | concentration |
| DNA template | 43 ng/μL | 46.27 μL | 40 ng/μL |
| Biotin-dPEG11-oxyamine | 10 mM | 2.48 μL | 0.5 mM |
| HCl | | | |
| MES buffer | 200 mM, pH 6.0 | 1.25 μL | 5 mM |
| Total volume | | 50 μL | |

### Example 2. Verification of Specificity and Efficiency of G Base-Specific Reactions

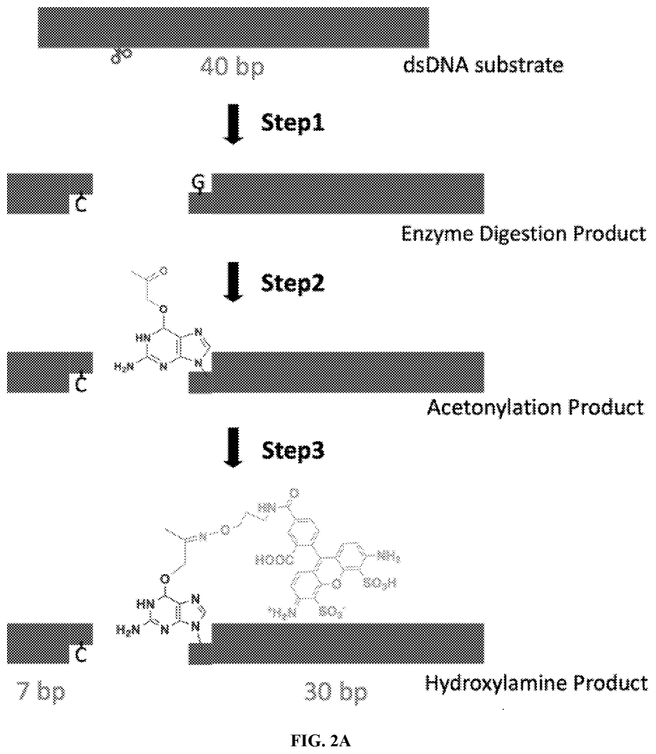

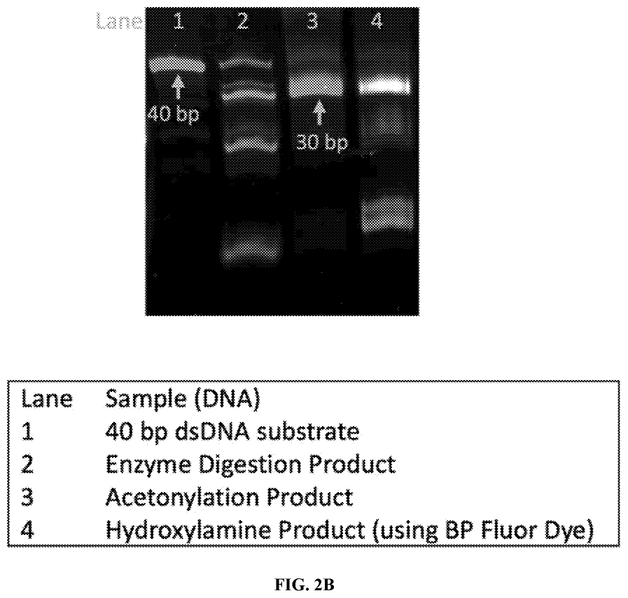

The acetonylation and oxime formation reactions on short single-stranded DNA (20 nt) and double-stranded DNA (40 bp) were first studied. These reactions were designed to specifically label a green fluorescent dye (BP Fluor 488 Hydroxylamine dye) onto the overhang terminal G base, with nearly 100% efficiency, as shown in FIGS. 2A-2B. Lane 1 shows reference 40 bp oligo detected in the GelRed channel; Lane 2 shows raw enzyme digestion production before purification, with the second band from top being the 30 bp dsDNA with single-strand overhang containing G, all bands detected exclusively in the GelRed channel; Lane 3 shows 30 bp dsDNA after acetonylation, detected in the GelRed channel; Lane 4 shows 30 bp dsDNA after oxime formation to conjugate a green fluorescent dye, appearing as a superimposed signal originating from both the GelRed channel and the Alexa Fluor 488 channel. The results were analyzed using 20% PAGE, and the gel images were captured through two channels: the Fluorescent channel and the GelRed channel (see FIG. 2B). The overlaid image of the two channels clearly shows that only the sequence where both chemical reactions were performed exhibits green fluorescence (Lane 4, which originally appears as a green band in the merged image due to overlap with the GelRed signal), while control sequences showed no signal in the fluorescent channel.

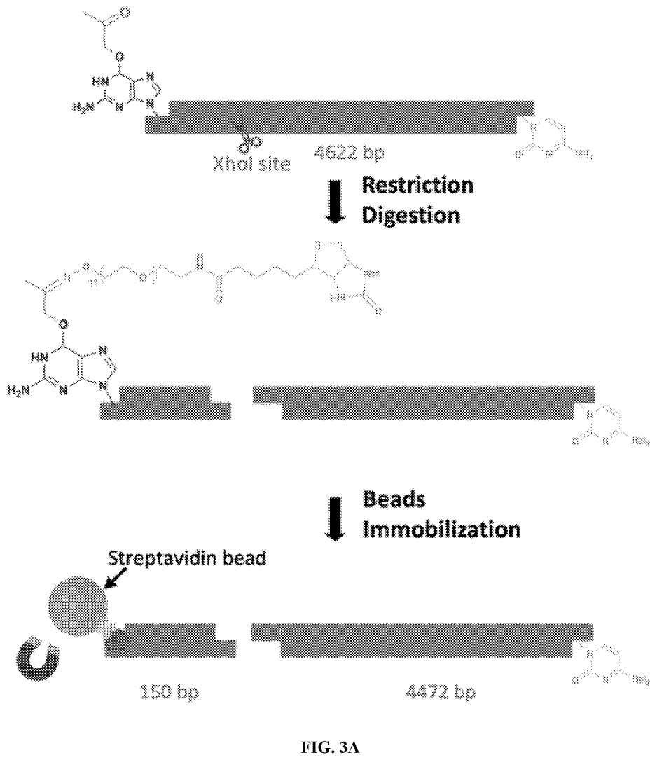

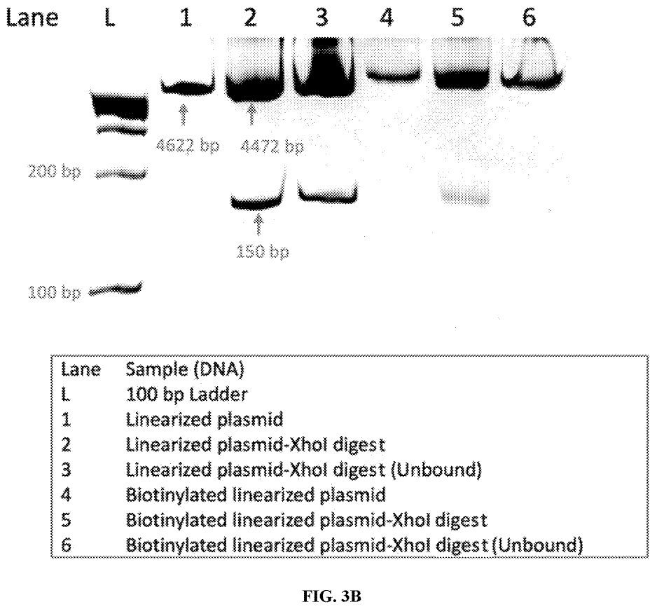

For linearized plasmid DNA, templates modified by acetonylation and oxime formation were subjected to XhoI restriction enzyme digestion (THERMO SCIENTIFIC™). This digestion produced two fragments: a 150 bp fragment containing the exposed G base and a 4472 bp fragment containing the C base. The digested fragments were subject to biotinylation and then incubated with DYNABEADS™ MYONE™ Streptavidin C₁ magnetic beads (INVITROGEN™), as illustrated in FIG. 3A.

According to the native PAGE gel electrophoresis results shown in FIG. 3B, the 150 bp fragment was completely removed from the unbound solution after bead treatment (Lane 6), indicating successful ligation of the G base-modified fragment to the beads. In contrast, the 150 bp fragment remained in the unbound solution for the control sample (Lane 3 vs. Lane 2). These results confirm the specificity and efficiency of the one-pot chemical reaction, which precisely targets the exposed G bases on the linearized plasmid DNA.

### Example 3. Verification of Attachment of Reaction Products to Streptavidin Beads

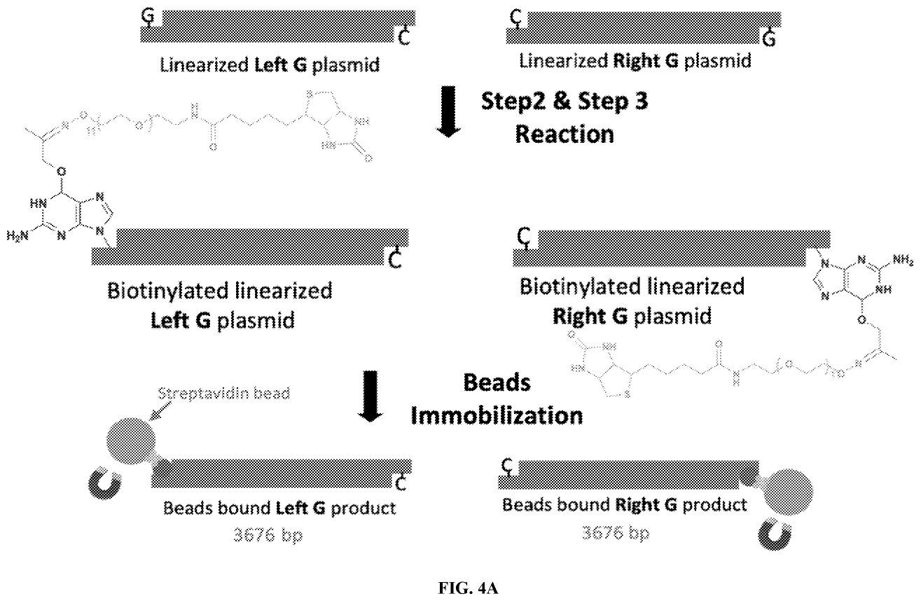

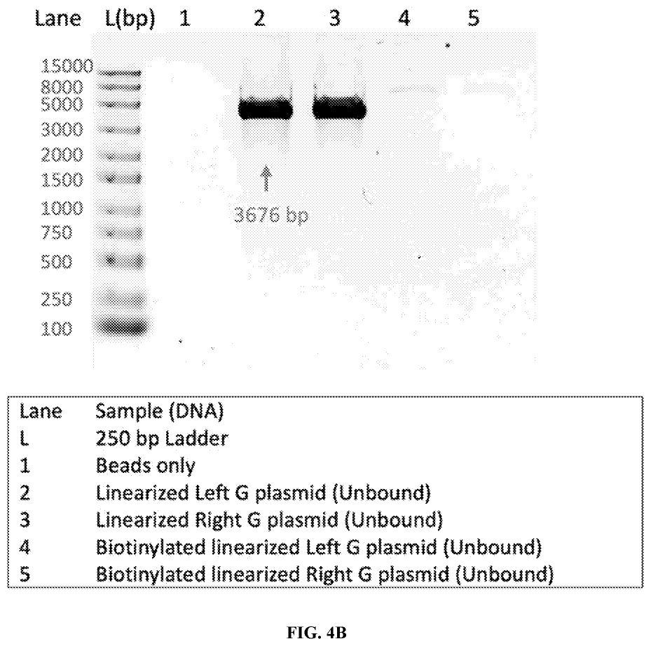

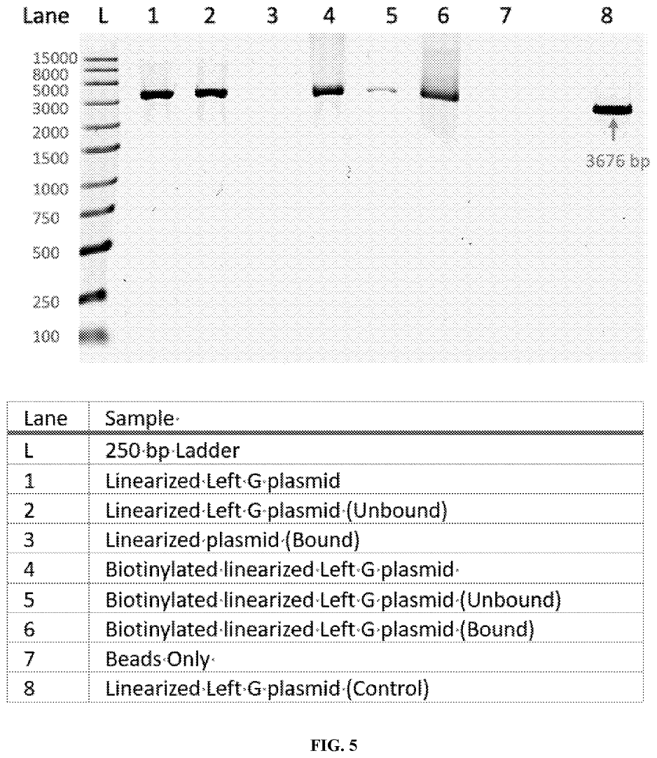

To validate that the chemically modified DNA templates can successfully bind to streptavidin-coated beads, two types of beads were tested: DYNABEADS™ M-280 Streptavidin beads (used in FIGS. 4A-4B) and DYNABEADS™ Streptavidin beads for IVT (THERMO SCIENTIFIC™) (used in FIG. 5) with similar experimental procedures, including a design strategy for site-specific terminal biotinylation and immobilization of the modified linearized plasmids on magnetic beads as illustrated in FIG. 4A, followed by agarose gel electrophoresis to verify the binding results. The binding experiments followed the manufacturer's protocols, using 3676-bp linearized plasmid DNA templates containing a single-nucleotide G overhang at one terminus (including both Linearized Left G and Linearized Right G plasmids in FIGS. 4A-4B, and the Linearized Left G plasmid in FIG. 5).

As shown in FIGS. 4A-4B and FIG. 5, after binding, the band intensities of the linearized plasmid DNA in the unbound solutions significantly decreased for both types of beads. This confirms that the chemically modified DNA templates successfully attached to the streptavidin-coated beads. These results demonstrate that the chemical reaction produces DNA templates that are compatible with bead binding, regardless of the differences in bead properties.

### Example 4. Verification of Integrity of Plasmid DNA and High-Quality In Vitro Transcription

The bead-bound DNA templates (i.e., the test sample obtained in FIG. 5 (lane 6)) were tested for IVT efficiency using the MEGASCRIPT™ T7 Transcription Kit (INVITROGEN™). The reaction included ATP, CTP, UTP, and GTP in a molar ratio of 1:1:1:1 (TriLink BioTechnologies). After TURBO™ DNase (INVITROGEN™) and Antarctic phosphatase (NEW ENGLAND BIOLABS®) treatments, the synthesized mRNA was purified using the MONARCH® RNA Cleanup Kit (NEW ENGLAND BIOLABS®).

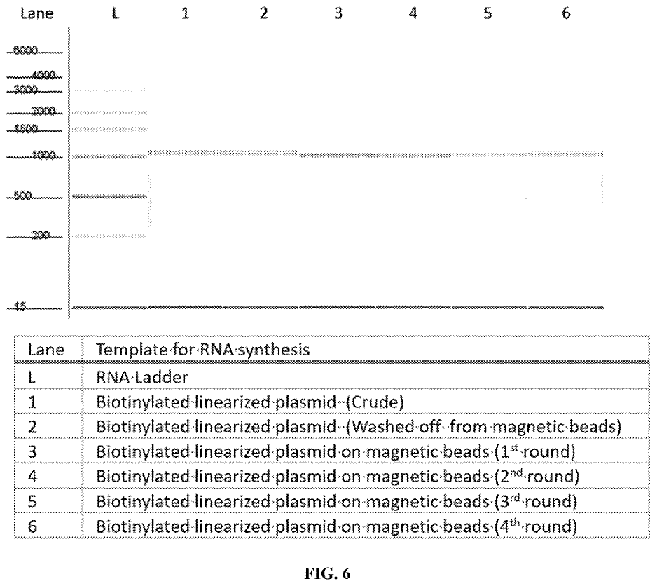

The quality of the mRNA was analyzed using a Fragment Analyzer (AGILENT®). As shown in FIG. 6, the bead-bound DNA templates successfully produced high-quality target mRNA. Additionally, the bead-template complexes were reused for multiple in vitro transcription cycles while maintaining consistent mRNA synthesis efficiency. This demonstrates the robustness and reusability of the bead-bound DNA templates, offering a cost-effective and efficient solution for mRNA production workflows.

What is claimed is:

1. A method for site-selective terminal modification of a double-stranded nucleic acid molecule, comprising
(a) providing a double-stranded nucleic acid molecule comprising at least one single-stranded terminal overhang containing a guanine base or a guanine analog base;
(b) contacting the double-stranded nucleic acid molecule with a carbonyl-introducing agent to provide an intermediate nucleic acid molecule comprising a carbonyl functional group at the guanine base or guanine analog base; and
(c) contacting the intermediate nucleic acid molecule with an oxime-forming nucleophile to form a modified double-stranded nucleic acid molecule comprising an oxime linkage at the guanine base or guanine analog base.

2. The method of claim 1, wherein the carbonyl-introducing agent has a structure of Formula I: R₁—CO—R₂, wherein R₁ and R₂ are independently selected from the group consisting of C₁-C₂₀ alkyl, C₂-C₂₀ alkenyl, C₃-C₁₂ cycloalkyl, C₁-C₂₀ epoxyalkyl, C₁-C₂₀ diazoalkyl, C₁-C₂₀ haloalkyl, aryl, heteroaryl, and aralkyl; and
wherein at least one of R₁ and R₂ contains a leaving group selected from the group consisting of diazo, halogen, an epoxide group, and an activated Michael acceptor group.

3. The method of claim 2, wherein the carbonyl-introducing agent is selected from the group consisting of α-diazocarbonyl compounds, phenacyl reagents, activated enone reagents, formylation reagents capable of installing a pendant aldehyde at the guanine base or guanine analog base, and aldehyde-transfer reagents capable of generating a pendant aldehyde at the guanine base or guanine analog base.

4. The method of claim 2, wherein one of R₁ and R₂ is selected from C₁-C₆ alkyl, and the other is selected from C₁-C₆ diazoalkyl.

5. The method of claim 1, wherein the oxime-forming nucleophile has a structure of Formula II: R₃—O—NH—R₄, wherein R₃ and R₄ are independently selected from the group consisting of H, C₁-C₂₀ alkyl, C₁-C₂₀ haloalkyl, C₁-C₂₀ hydroxyalkyl, C₁-C₂₀ aminoalkyl, C₁-C₂₀ alkoxyalkyl, aryl, heteroaryl, a detectable label, an affinity tag, a polymer, a lipid, a solid support, an oligonucleotide, a reactive functional group, and pharmaceutically acceptable salts thereof.

6. The method of claim 5, wherein the oxime-forming nucleophile is a hydroxylamine-containing compound or an oxyamine-functionalized ligand,
wherein the hydroxylamine-containing compound is selected from the group consisting of hydroxylamine, substituted hydroxylamines, O-substituted hydroxylamines, and pharmaceutically acceptable salts thereof; and
wherein the oxyamine-functionalized ligand is selected from the group consisting of oxyamine-functionalized biotin derivatives, oxyamine-functionalized fluorophores, oxyamine-functionalized PEG reagents, oxyamine-functionalized polymers, oxyamine-functionalized lipids, oxyamine-functionalized oligonucleotides, oxyamine-functionalized solid supports, and pharmaceutically acceptable salts thereof.

7. The method of claim 5, wherein the oxime-forming nucleophile is an O-substituted hydroxylamine, wherein R₃ is selected from the group consisting of C₁-C₂₀ alkyl, C₁-C₂₀ haloalkyl, C₁-C₂₀ hydroxyalkyl, C₁-C₂₀ aminoalkyl, PEG, aryl, heteroaryl, and an affinity tag, and wherein R₄ is H.

8. The method of claim 5, wherein the oxime-forming nucleophile is an aminooxy functionalized ligand selected from the group consisting of aminooxy-functionalized biotin derivatives, aminooxy-functionalized fluorophores, aminooxy-functionalized PEG reagents, aminooxy-functionalized polymers, aminooxy-functionalized lipids, aminooxy-functionalized oligonucleotides, aminooxy-functionalized solid supports, and pharmaceutically acceptable salts thereof.

9. The method of claim 5, wherein the oxime-forming nucleophile is hydroxylamine, Biotin-dPEG₁₁-oxyamine HCl, or BP FLUOR 488 Hydroxylamine, and wherein the carbonyl introducing agent is diazoacetone.

10. The method of claim 1, wherein step (a) comprises providing a double-stranded nucleic acid molecule comprising two single-stranded terminal overhangs, wherein only one of the terminal overhangs contains a guanine base or a guanine analog base, and
wherein the guanine analog base is a guanine derived or guanine related nucleobase capable of undergoing carbonyl installation and oxime formation, which is selected from the group consisting of 8-oxo-guanine, inosine, xanthine, 2-aminopurine, 7-deazaguanine, 8-bromoguanine, 6-thioguanine, O6-benzyl-guanine, 8-azido-guanine, iso-guanine, Z base, and any combination thereof.

11. The method of claim 9, wherein step (c) comprises attaching the oxime-forming nucleophile to a solid support selected from the group consisting of magnetic beads, polymeric beads, membranes, and microarray surfaces, prior to contacting the oxime-forming nucleophile with the intermediate nucleic acid molecule.

12. The method of claim 4, wherein the oxime-forming nucleophile is part of a bifunctional linker comprising an aminooxy group and a second reactive group selected from the group consisting of azide, alkyne, maleimide, NHS ester, and thiol; or
wherein the double-stranded nucleic acid molecule is selected from the group consisting of DNA, cDNA, RNA, Locked Nucleic Acid (LNA), DNA/RNA hybrid, and derivatives thereof.

13. The method of claim 1, wherein step (a) comprises generating the single-stranded terminal overhang by an enzymatic method comprising digesting the double-stranded nucleic acid molecule with one or more restriction enzymes to generate at least one single-stranded terminal overhang containing an exposed guanine base or guanine analog base.

14. The method of claim 13, wherein the one or more restriction enzymes are selected from the group consisting of Type II restriction enzymes, Type IIP restriction enzymes, Type IIS restriction enzymes, Type IIG restriction enzymes, Type III restriction enzymes, and any combination thereof.

15. A modified double-stranded nucleic acid molecule obtained by the method of claim 1.

16. The modified double-stranded nucleic acid molecule of claim 15 comprising a nucleic acid template configured for in vitro transcription (IVT).

17. A method for fluorescent labeling, protein-DNA conjugation, directional surface immobilization, biosensing, nanotechnology applications, or terminally controlled DNA cyclization or patterning, comprising applying the modified double-stranded nucleic acid molecule of claim 15.

18. A kit for implementing the method for site-selective terminal modification of a double-stranded nucleic acid molecule according to claim 1, the kit comprising: a carbonyl-introducing agent; and an oxime-forming nucleophile.

19. The kit of claim 18, further comprising one or more restriction enzymes, wherein the restriction enzymes are capable of digesting the nucleic acid to generate at least one single-stranded terminal overhang containing at least one exposed guanine base or guanine analog base; or wherein the kit further comprises a double-stranded nucleic acid molecule that is digestible by one or more restriction enzymes to generate one or two single-stranded terminal overhangs, wherein only one or both of the terminal overhangs comprise a guanine base or guanine analog base.

20. The kit of claim 19, wherein the one or more restriction enzymes are selected from the group consisting of Type II restriction enzymes, Type IIP restriction enzymes, Type IIS restriction enzymes, Type IIG restriction enzymes, Type III restriction enzymes, and any combination thereof.
